(19) United States
(12) Patent Application Publication
(10) Pub. No.: US 2025/0332445 A1
(43) Pub. Date: Oct. 30, 2025

(54) CANCER THERAPY DELIVERY ASSEMBLY

(71) Applicant: Boston Scientific Scimed, Inc., Maple Grove, MN (US)

(72) Inventors: Patrick A. Haverkost, Crystal, MN (US); Joel N. Groff, Delano, MN (US); Scott McGhee, Ottawa (CA); David Raab, Roseville, MN (US); McKenzie Dudley, St. Louis Park, MN (US)

(21) Appl. No.: 19/188,282

(22) Filed: Apr. 24, 2025

Related U.S. Application Data
(60) Provisional application No. 63/638,817, filed on Apr. 25, 2024.

(51) Int. Cl. A61N 5/10; A61K 51/12; A61M 25/02

(52) U.S. Cl. CPC A61N 5/1007; A61K 51/1255; A61M 25/02; A61K 2121/00; A61M 2209/082; A61M 2209/084; A61N 2005/1021; A61N 2005/1094

(57) ABSTRACT

Embodiments herein relate to a cancer therapy delivery system including a cancer therapy delivery assembly or box with adjustment features. In an embodiment, a cancer therapy delivery assembly includes a base, an inner housing disposed over the base, and an outer housing disposed over the inner housing. The outer housing is moveable along a vertical axis relative to the inner housing. The assembly can also include a rotatable component holder disposed on the outer housing and configured to rotate relative to the outer housing. The assembly can also include a transparent shielding member disposed over the rotatable component holder and a catheter support arm attached to the rotatable component holder. The catheter support arm can be configured to adjustably move inward or outward with respect to a center of the cancer therapy delivery assembly. Other embodiments are also included herein.

This application claims the benefit of U.S. Provisional Application No. 63/638,817, filed Apr. 25, 2024, the content of which is herein incorporated by reference in its entirety.

### FIELD

Embodiments herein relate to cancer therapy delivery systems. M ore specifically, embodiments herein relate to a cancer therapy delivery system including a cancer therapy delivery assembly or box with adjustment features.

### BACKGROUND

According to the American Cancer Society, cancer accounts for nearly 25% of the deaths that occur in the United States each year. Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division.

Surgery is a common first-line therapy for many cancerous tumors. However, not every tumor can be surgically removed. Chemotherapy and immunotherapy are other common therapeutic approaches but can include substantial side effects. The use of radiation represents another approach. Specifically, radiation therapy aims at damaging the DNA of cancer cells so that they lose the capability to divide and proliferate, thus leading to the cell death process for the cancerous cells.

### SUMMARY

Embodiments herein relate to a cancer therapy delivery system including a cancer therapy delivery assembly or box with adjustment features. In a first aspect, a cancer therapy delivery assembly can be included having a base, an inner housing, and an outer housing. The inner housing can be disposed over the base and the outer housing can be disposed over the inner housing. The outer housing can be vertically adjustable along a vertical axis relative to the inner housing to allow vertical height adjustment. A rotatable component holder can be included and can be disposed on the outer housing. The rotatable component holder can be configured to rotate relative to the outer housing. A transparent shielding member can be included that can be disposed over the rotatable component holder.

In a second aspect, in addition to one or more of the preceding or following aspects, or in the alternative to some aspects, the system can further include a vertical position adjustment device, wherein manipulation of the vertical position adjustment device causes the outer housing to move along a vertical axis relative to the inner housing.

In a third aspect, in addition to one or more of the preceding or following aspects, or in the alternative to some aspects, the vertical position adjustment device can include an adjustment wheel.

In a fourth aspect, in addition to one or more of the preceding or following aspects, or in the alternative to some aspects, can further include a rotational lock element, wherein the rotational lock element can be configured to lock the rotatable component holder in a particular rotational position.

In a fifth aspect, in addition to one or more of the preceding or following aspects, or in the alternative to some aspects, the inner housing can include a top central aperture, the outer housing can include a top central aperture, and the rotatable component holder can include a cylindrical base, wherein the cylindrical base can be configured to pass through the top central aperture of the inner housing and the top central aperture of the outer housing.

In a sixth aspect, in addition to one or more of the preceding or following aspects, or in the alternative to some aspects, the rotatable component holder can further include a top platform, wherein the top platform can be attached to a top of the cylindrical base and extends radially outward from the same.

In a seventh aspect, in addition to one or more of the preceding or following aspects, or in the alternative to some aspects, the top platform can be configured to rest upon a top of the outer housing. The cancer therapy delivery assembly can further include a catheter support arm, wherein the catheter support arm can be attached to the rotatable component holder. The catheter support arm can be configured to adjustably move inward or outward with respect to a center of the cancer therapy delivery assembly.

In an eighth aspect, in addition to one or more of the preceding or following aspects, or in the alternative to some aspects, the catheter support arm can include a catheter attachment element, wherein the catheter attachment element can be disposed on an end of the catheter support arm.

In a ninth aspect, in addition to one or more of the preceding or following aspects, or in the alternative to some aspects, the rotatable component holder can define a well, wherein the well can be configured to receive a vial holder and a vial with radioactive microspheres.

In a tenth aspect, in addition to one or more of the preceding or following aspects, or in the alternative to some aspects, the outer housing can include one or more exterior facets.

In an eleventh aspect, in addition to one or more of the preceding or following aspects, or in the alternative to some aspects, the outer housing can include a substantially polygonal perimeter in cross-section.

In a twelfth aspect, in addition to one or more of the preceding or following aspects, or in the alternative to some aspects, the outer housing can be disposed over the inner housing in a nested configuration.

In a thirteenth aspect, in addition to one or more of the preceding or following aspects, or in the alternative to some aspects, the transparent shielding member can define one or more slots to allow passage of a fluid conduit therethrough.

In a fourteenth aspect, a cancer therapy delivery assembly can be included having a base, an inner housing disposed over the base, and an outer housing disposed over the inner housing. The outer housing can be moveable along a vertical axis relative to the inner housing. A transparent shielding member can be included and can be disposed over the outer housing. A vertical position adjustment device can be included wherein manipulation of the vertical position adjustment device causes the outer housing to move along a vertical axis relative to the inner housing.

In a fifteenth aspect, in addition to one or more of the preceding or following aspects, or in the alternative to some aspects, the outer housing defining a well, wherein the well can be configured to receive a vial holder and a vial with radioactive microspheres.

In a sixteenth aspect, in addition to one or more of the preceding or following aspects, or in the alternative to some aspects, the outer housing can include one or more exterior facets.

In a seventeenth aspect, in addition to one or more of the preceding or following aspects, or in the alternative to some aspects, the outer housing can include a substantially polygonal perimeter in cross-section.

In an eighteenth aspect, in addition to one or more of the preceding or following aspects, or in the alternative to some aspects, the outer housing can be disposed over the inner housing in a nested configuration.

In a nineteenth aspect, in addition to one or more of the preceding or following aspects, or in the alternative to some aspects, the assembly can further include a catheter support arm, wherein the catheter support arm can be attached to the outer housing and wherein the catheter support arm can be configured to adjustably move inward or outward with respect to a center of the cancer therapy delivery assembly.

In a twentieth aspect, a method of delivering a cancer therapy can be included. The method can include positioning a catheter attachment element. Positioning can include moving an outer housing vertically with respect to an inner housing, extending a catheter support arm radially, and rotating a rotatable component holder with respect to the outer housing. The method can also include attaching a cancer therapy delivery catheter to the catheter attachment element, and delivering therapeutic radioactive microspheres to a patient via the delivery catheter.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic diagram of components of an exemplary cancer therapy delivery system in accordance with various embodiments herein.

FIG. 2 is a perspective view of cancer therapy delivery assembly in accordance with various embodiments herein.

FIG. 3 is an exploded view of cancer therapy delivery assembly in accordance with various embodiments herein.

FIG. 4 is a perspective view of an inner housing in accordance with various embodiments herein.

FIG. 5 is a perspective view of an outer housing in accordance with various embodiments herein.

FIG. 6 is a perspective view of a rotatable component holder in accordance with various embodiments herein.

FIG. 7 is a perspective view of a mounting bracket in accordance with various embodiments herein.

FIG. 8 is a side view of a mounting bracket in accordance with various embodiments herein.

FIG. 9 is a perspective view of cancer therapy delivery assembly in accordance with various embodiments herein.

FIG. 10 is a perspective view of cancer therapy delivery assembly in accordance with various embodiments herein.

FIG. 11 is a flow diagram illustrating operations of a method in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Radiation therapy aims at damaging the DNA of cancer cells so that they lose the capability to divide and proliferate, thus leading to the cell death process for the cancerous cells. Brachytherapy is a form of radiation therapy where a sealed radiation source is placed inside or next to the area requiring treatment. As one form of brachytherapy, targeted radioembolization therapy can be used to treat unresectable tumors. For example, Y-90 glass microspheres can be delivered into or adjacent to a tumor through a microcatheter placed into an artery that supplies blood to the tumor. The beta radiation emitted by the Y-90 can exert a local radiotherapeutic effect on the tumor. Other radioisotopes can also be used in some types of brachytherapy.

Therapeutic microspheres or other particulates can be delivered to the patient through a catheter or other delivery line, carried along by a flow of carrier fluid onto a target site in the body. It can be important to be able to position the proximal end of a catheter or other delivery line as desired by the clinician and able to fix the same in place. It can also be important to be able to keep multiple components of the system together in a convenient assembly. It can also be important to provide a shield against beta radiation emitted by the therapeutic microspheres to protect the clinician and/or others.

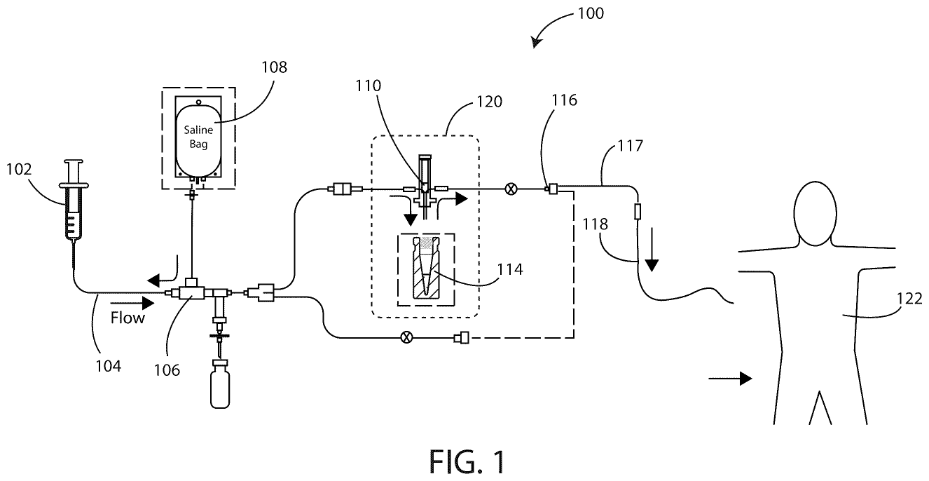

Embodiments herein include a cancer therapy delivery assembly or box with adjustment features that work as part of an overall cancer therapy delivery system. Referring now to FIG. 1, a schematic diagram of components of an exemplary cancer therapy delivery system is shown in accordance with various embodiments herein. Major parts of the cancer therapy delivery system 100 include a therapeutic fluid delivery device 102 (or pump, which in some cases can take the form of a syringe or syringe-like device), a fluid supply tube 104 or line, and a flow control valve 106. In this example, the cancer-therapy delivery system 100 also includes a saline supply reservoir 108 (or fluid reservoir). The saline can serve as the carrier fluid to be mixed with the microspheres. The saline solution can be at various concentrations such as (0.3%, 0.5%, 0.7%, 0.9%, or the like). In some embodiments, the carrier fluid (e.g., a saline solution) can also include one or more other components. For example, in some embodiments the carrier fluid can include heparin (in the case of saline, a heparinized saline solution). The cancer-therapy delivery system 100 can also include a fluid injector and withdrawal assembly 110. The cancer-therapy delivery system 100 can also include a radioactive microsphere supply reservoir/mixing chamber 114.

The fluid injector and withdrawal assembly 110 and the radioactive microsphere supply reservoir/mixing chamber 114 can be within a cancer therapy delivery assembly 120 (or box), which can aid in positioning the proximal end of a microcatheter or other delivery line as desired by the clinician and able to fix the same in place, keeping multiple components of the system (e.g., 110 and 114) together in a convenient assembly, and providing a shield against beta radiation emitted by the therapeutic microspheres to protect the clinician and/or others. Further details of exemplary cancer therapy delivery assemblies 120 or boxes are described in greater detail below.

The carrier fluid can be conveyed into the microsphere supply reservoir/mixing chamber 114 via the fluid injector and withdrawal assembly 110 (such as via a needle, cannula, or tube serving as an inflow conduit) and then the carrier fluid mixes with the microspheres therein forming a mixture or suspension before passing out through the fluid injector and withdrawal assembly 110 (such as via a needle, cannula, or tube serving as an outflow conduit).

In this example, the cancer-therapy delivery system 100 also includes an outflow port 116. The cancer-therapy delivery system 100 also includes a microcatheter 118. The mixture or suspension of microspheres flows from an outflow conduit of the fluid injector and withdrawal assembly 110 and then through the outflow port 116 and into the microcatheter 118. FIG. 1 also shows a patient 122 into which the microcatheter 118 can be inserted to deliver the therapeutic suspension of microspheres.

In some embodiments, the microcatheter 118 can specifically be one with a relatively small diameter, such as a microcatheter with a neuro use indication (hereafter “neurocatheter”). In some embodiments, the microcatheter 118 outer diameter can be as small as 0.33 millimeters (mm) (0.013 inches) or less. However, in other embodiments the catheter or microcatheter can be larger in diameter. In some embodiments, the catheter or microcatheter can have a diameter of less than or equal to 5.33, 4.32, 4.01, 3.66, 3.33, 3.00, 2.67, 2.34, 2.01, 1.68, 1.35, 0.99, 0.66, or even 0.33 mm (0.288, 0.21, 0.17, 0.158, 0.144, 0.131, 0.118, 0.105, 0.092, 0.079, 0.066, 0.053, 0.039, 0.026, or even 0.013 inches (equivalent to 1 Fr)), or a diameter falling within a range between any of the foregoing.

While not intending to be bound by theory, inner diameters greater than a certain point can lead to undesirable microsphere dropout. As such, in various embodiments herein, the inner diameter of the microcatheter (or the inner diameter of a fluid passage within the catheter) can be quite small. For example, in some embodiments, the microcatheter 118 inner diameter can be less than or equal to 0.050, 0.045, 0.040, 0.035, 0.030, 0.035, 0.020, or 0.015 inches (1.27, 1.143, 1.016, 0.889, 0.762, 0.508, or 0.381 mm), or a size falling within a range between any of the foregoing.

In use, various operations can be performed to prepare the system 100. For example, operations can be formed such as system priming, air/bubble removal, flushing operations, and the like. Then (omitting some possible operations for ease of explanation) the clinician or other system user can pull back on a plunger or similar mechanism of therapeutic fluid delivery device 102 causing fluid (such as saline) to be withdrawn from the saline supply reservoir 108, through the flow control valve 106 and the fluid supply tube 104, and into the fluid delivery device 102. Then the clinician or other system user can depress the plunger causing fluid to flow from the therapeutic fluid delivery device 102, through the fluid supply tube 104 (or fluid line), through the flow control valve 106, and into the fluid injector and withdrawal assembly 110. The fluid injector and withdrawal assembly 110 can be in fluid communication with the mixing chamber 114 and can direct a flow of fluid into the mixing chamber 114 coming from the therapeutic fluid delivery device 102 or pump such as through one of a pair of needles, cannulas, or tubes serving as an inflow conduit. The fluid can become mixed with microspheres in the mixing chamber 114 forming a suspension which can then exit via the fluid injector and withdrawal assembly 110 via another needle, cannula, or tube serving as an outflow conduit and through tubing and out of the outflow port 116 and into the microcatheter 118 and into a desired site of the patient 122. In some embodiments, the flow rate of fluid through the system can be less than or equal to about 25, 20, 25, 10, 8, 6, or 5 milliliters/minute (mL/min), or a flow rate falling within a range between any of the foregoing. For example, in some embodiments, the flow rate can be from 5 to 20 mL/min. After an initial volume of fluid is passed through to the patient this way, one or more flushes can be performed (e.g., additional amounts of carrier fluid can be run through the system and to the patient to ensure that all or nearly all of the microspheres are delivered to the patient).

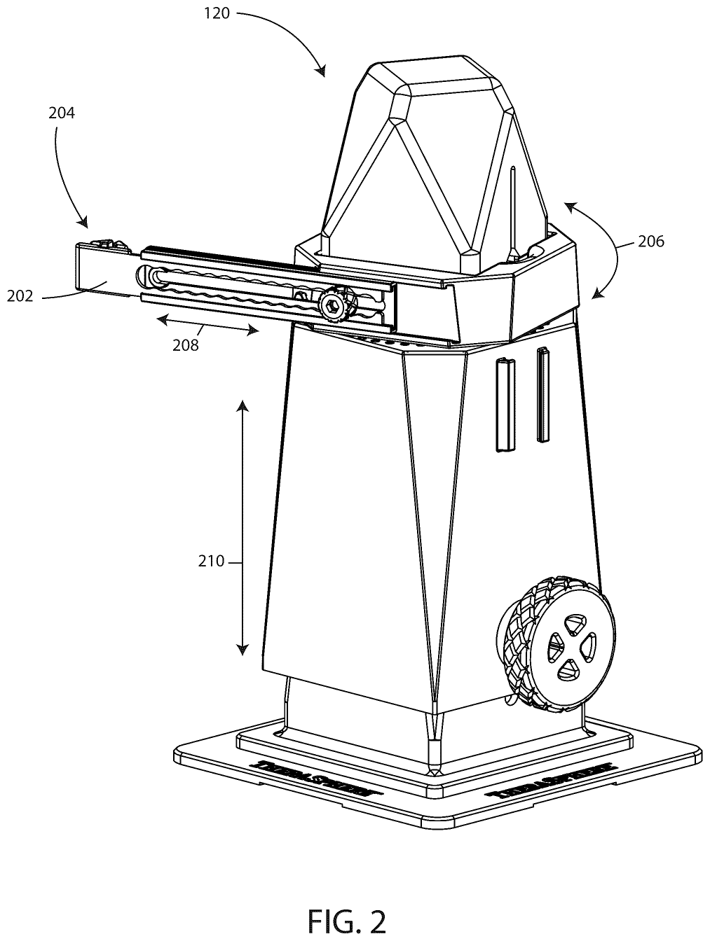

Referring now to FIG. 2, a perspective view of a cancer therapy delivery assembly 120 (or box) is shown in accordance with various embodiments herein. The cancer therapy delivery assembly 120 includes a catheter support arm 202. The catheter support arm 202 includes a catheter attachment element 204, which can be used to secure a delivery catheter or delivery line thereto. The catheter attachment element 204 can include various mechanisms to secure the delivery catheter thereto including, for example, a band, a strap, a snap-fit or friction-fit mechanism, an adhesive, or the like.

The cancer therapy delivery assembly 120 and/or portions thereof can be configured to rotate 206, which allows the catheter support arm 202 to also rotate. In some embodiments, rotation can be a full 360 degrees or more, but in other embodiments rotation can be limited to less than 360 degrees. Such rotation can allow the catheter attachment element 204 (and thereby the catheter attached thereto) to be rotated into a desired position.

The cancer therapy delivery assembly 120 can also be configured to allow vertical movement 210, which allows the catheter attachment element 204 to move up and down vertically. In some embodiments, the vertical movement 210 can be from 0 to 10, 15, 20, 25, 30, 35, 40, or 45 centimeters, or more (0 to 3.93, 5.91, 7.87, 9.84, 11.81, 13.78, 15.75, 17.72 inches, or more). The catheter support arm 202 can also extend 208 inward and outward relative to a center of the cancer therapy delivery assembly 120, which can allow inward and outward movement of the catheter attachment element 204, which can be used to secure a delivery catheter or delivery line thereto. In some embodiments, the catheter support arm 202 can telescope inward and outward. In some embodiments, the catheter support arm 202 can extend inward and outward linearly. In some embodiments, the catheter support arm 202 can slide inward and outward. Taken together, the movement afforded by the cancer therapy delivery assembly 120 allows a clinician to precisely position the catheter attachment element 204 (and thereby the delivery catheter or delivery line attached thereto) as desired.

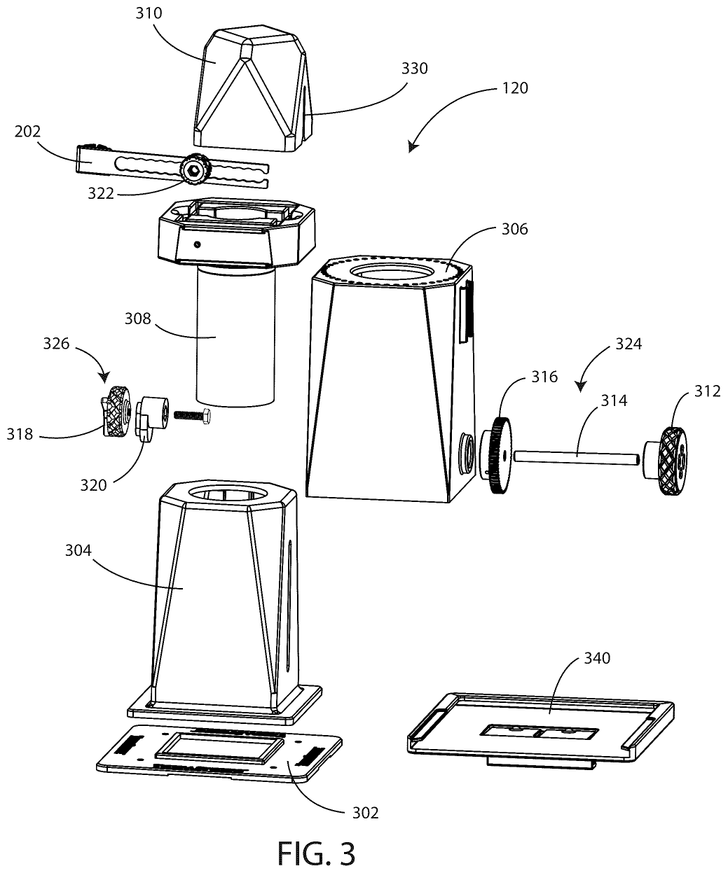

Referring now to FIG. 3, an exploded view of cancer therapy delivery assembly 120 is shown in accordance with various embodiments herein. As before, the cancer therapy delivery assembly 120 includes a catheter support arm 202. The catheter support arm 202 can be formed of various materials including, for example, metals, polymers, composites, ceramics, and the like. The catheter support arm 202 can be attached to a rotatable component holder 308. In various embodiments, the rotatable component holder 308 can be formed of a molded or otherwise formed (such as additive manufacturing, or the like) polymer. However, the rotatable component holder 308 could also be formed of other materials. Further aspects of the rotatable component holder 308 will be provided in greater detail below.

The catheter support arm 202 can be manually extended inward and outward relative to rotatable component holder 308. However, in some embodiments, extension could also be powered, such as with a linear actuator or the like. After the catheter support arm 202 is moved into a desired position, it can be secured in place using an arm fastener 322, such as a nut, bolt, screw, pin, detent, or the like.

The cancer therapy delivery assembly 120 also includes a base 302. The base 302 can be disposed at the bottom of the cancer therapy delivery assembly 120. The base 302 can be wider than other components of the cancer therapy delivery assembly 120 to provide stability. In some embodiments, the base 302 can be configured to be set upon a flat surface, such as the top surface of a cart, a table, or the like. The base can be formed of various materials including, but not limited to, polymers, metals, composites, glasses, ceramics, and the like. In some embodiments, the base 302 can secured to an underlying surface or other structure.

In various embodiments, an inner housing 304 can be disposed over a base 302. In some embodiments, the inner housing 304 can be fastened to the base 302. The inner housing 304 can be formed of various materials including, but not limited to polymers, metals, composites, and the like. Further aspects of the inner housing 304 will be provided in greater detail below.

The cancer therapy delivery assembly 120 can also include an outer housing 306. The outer housing 306 can be disposed over the inner housing 304. The outer housing 306 can be formed of various materials including, but not limited to polymers, metals, composites, and the like. The outer housing 306 can interface with both the inner housing 304 and the rotatable component holder 308. In some embodiments, the outer housing 306 can be disposed over the inner housing 304 in a nested configuration. In various embodiments, the rotatable component holder 308 can be disposed on the outer housing 306. In various embodiments, the rotatable component holder 308 can be configured to rotate relative to the outer housing 306. Further aspects of the outer housing 306 will be provided in greater detail below.

As referenced above, the cancer therapy delivery assembly 120 can be configured to allow vertical height adjustment to support positioning a proximal end of a delivery catheter or delivery line at a desired height. In various embodiments, the outer housing 306 can be moveable along a vertical axis relative to the inner housing 304 to effectuate positioning the delivery catheter at a desired height. The cancer therapy delivery assembly 120 can include a vertical position adjustment device 324 to facilitate such vertical movement. The vertical position adjustment device 324 can include adjustment knob or wheel 312 (or other user manipulatable element), gear 316, and adjustment shaft 314 to convey force from the adjustment knob or wheel 312 to the gear 316. In operation, the device user or clinician can turn the adjustment knob or wheel 312 causing the adjustment shaft 314 and the gear 316 connected thereto to turn. In some embodiments, the gear 316 can interface with a rack or similar structure which can be attached to the inner housing 304, causing vertical movement of the outer housing 306 relative to the inner housing 304 (up or down).

In some embodiments, the cancer therapy delivery assembly 120 can also include a rotational lock element 326. In various embodiments, the rotational lock element 326 can be configured to lock a rotatable component holder 308 in a particular rotational position. In this embodiment, the rotational lock element 326 includes a lock wheel 318 and a lock mechanism 320. In some embodiments, when the lock mechanism 320 is engaged it can physically interface with a portion of the rotatable component holder 308 or other rotating element to prevent further rotation from occurring. However, it will be appreciated that there are many forms that the rotational lock element 326 can take such as lock lever, a lock pin, a lock bolt or screw, or the like.

The cancer therapy delivery assembly 120 can also include a transparent shielding member 310. In various embodiments, the transparent shielding member 310 can be disposed over a rotatable component holder 308. However, in some embodiments, the transparent shielding member 310 can be disposed directly over the outer housing 306. The transparent shielding member 310 can function to provide a shield against beta radiation emitted by the therapeutic microspheres to protect the clinician and/or others. The transparent shielding member 310 can be formed of various materials that can be effective to block radiation as well as allow the user to see into the area beneath the transparent shielding member 310. For example, in some embodiments, the transparent shielding member 310 can be formed of a polymer (such as a transparent acrylic polymer like poly(methyl methacrylate) or another polymer). In some embodiments, if transparency is not necessary, then a lead or tungsten shielding member could be used.

The transparent shielding member 310 can include one or more apertures or slots to allow for the passage of a fluid conduit therethrough 330. In some embodiments, slots can be convenient as it can allow components (such as the mixing chamber, microsphere vial carrier and vial, the fluid injector and withdrawal assembly, etc.) to be positioned on the top of the other components of the cancer therapy delivery assembly 120 or box with fluid lines extending on either side and then the transparent shielding member 310 can be placed down over the top.

In various embodiments, a mounting bracket 340 can also be included. The mounting bracket can facilitate mounting of the cancer therapy delivery assembly to a hospital bed, such as through attachment to the frame, rails, posts, head board, foot board, or other parts thereof. In some embodiments, the mounting bracket 340 can facilitate mounting of the cancer therapy delivery assembly to a portion of a hospital bed meeting standard IEC 60601-2-52.

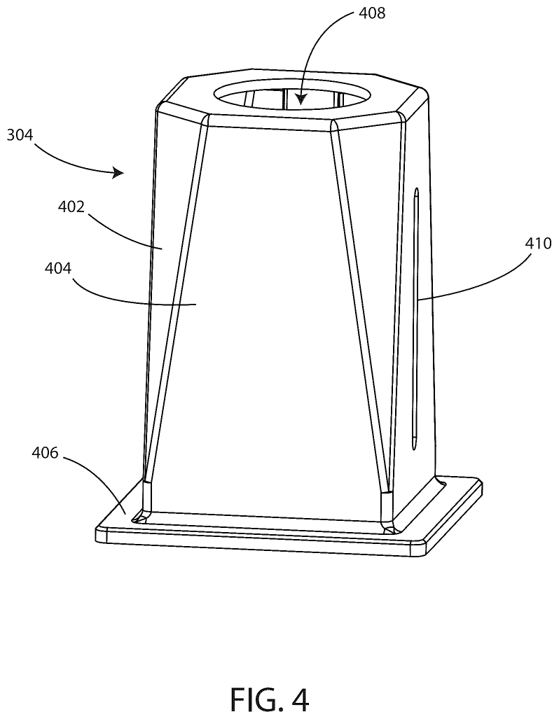

Referring now to FIG. 4, a perspective view of an inner housing 304 is shown in accordance with various embodiments herein. The inner housing 304 can be of various shapes, however, in this embodiment the inner housing 304 includes flat sides 404 or facets. The inner housing 304 can be substantially polygonal perimeter in cross-section.

In various embodiments, the inner housing 304 can be substantially hollow inside. In this embodiment, the inner housing 304 also includes corners 402 disposed between flat sides 404. In some embodiments, a portion of the corners 402 can be flattened and extend a portion of the overall vertical length of the inner housing 304. In this manner, when viewed in cross-section, the inner housing 304 can appear to have a greater number of sides (including the flattened corner portions) at the top of the inner housing 304 than at the bottom thereof. In some embodiments, the inner housing 304 can be tapered from bottom to top. This can be achieved in various ways. In this embodiment, the corners are triangular in shape and angled inward toward the center of the inner housing 304 and inverted such that the flat sides 404 are narrower at the top of the inner housing 304 than at the bottom.

In this embodiment, the inner housing 304 also includes a base flange 406. In some embodiments, the base flange 406 can be used to facilitate attachment of the inner housing 304 to the base 302. In some embodiments, the base flange 406 can be fastened to the base 302. However, in some embodiments the base flange 406 can be omitted.

The inner housing 304 can define a top central aperture 408. The top central aperture 408 can align with a similar aperture on the outer housing 306 as will be described below. The top central aperture 408 can be configured to receive a portion of rotatable component holder 308 as described below. In various embodiments, the top central aperture 408 is substantially circular to accommodate rotation of the rotatable component holder 308. However, in other embodiments, the top central aperture 408 can take on various other shapes.

In some embodiments, the inner housing 304 also includes a slot 410 (in some cases with a matching slot on the opposite side of the inner housing 304 not shown in the view forming a pair). In some cases, the slot 410 can be used to accommodate passage of adjustment shaft 314. For example, as the outer housing 306 is moved relative to the inner housing 304, the adjustment shaft 314, fixed to the outer housing 306, can move upward within the slot 410.

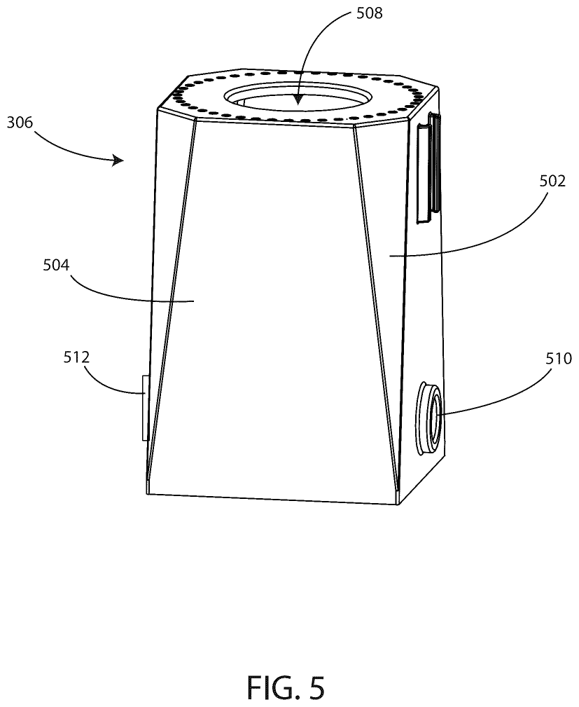

Referring now to FIG. 5, a perspective view of an outer housing 306 is shown in accordance with various embodiments herein. In this embodiment, the outer housing 306 also includes a one or more exterior faces 504 (or facets or flat portions) and corners 502. In some embodiments, a portion of the corners 502 can be flattened and extend a portion of the overall vertical length of the outer housing 306. In this manner, when viewed in cross-section, the outer housing 306 can appear to have a greater number of sides (including the flattened corner portions) at the top of the outer housing 306 than at the bottom thereof. In various embodiments, the outer housing 306 can be substantially polygonal perimeter in cross-section, in some cases matching a corresponding portion of the inner housing 304.

In various embodiments, the outer housing 306 can be substantially hollow inside. In some embodiments, the outer housing 306 can be tapered from bottom to top. This can be achieved in various ways. In this embodiment, the corners 502 are triangular in shape and angled inward toward the center of the outer housing 306 and inverted such that the exterior faces 504 are narrower at the top of the outer housing 306 than at the bottom.

In some embodiments, the exterior faces 504 (or facets or flat portions) and corners 502 can match with those on the inner housing 304 in order to facilitate the outer housing 306 fitting over the inner housing 304, such as in a nested configuration with a particular orientation of the inner housing 304 to the outer housing 306. In some embodiments, the presence of the exterior faces 504 (or facets or flat portions) and corners 502 can also facilitate gripping of the outer housing 306 by a user.

As with the inner housing 304, the outer housing 306 can also include a top central aperture 508. The top central aperture 508 can align with a similar aperture on the inner housing 304 when the outer housing 306 is fit over the inner housing 304. The top central aperture 508 can be configured to receive a portion of rotatable component holder 308. In various embodiments, the top central aperture 508 is substantially circular to accommodate rotation of the rotatable component holder 308. However, in other embodiments, the top central aperture 508 can take on various other shapes.

In this embodiment, the outer housing 306 also includes a first shaft aperture 510 or fitment and a second shaft aperture 512 or fitment, which can accommodate the adjustment shaft 314. In some embodiments other components can also be included to accommodate the to accommodate operation of the adjustment shaft 314 such as bushings, bearings, or the like.

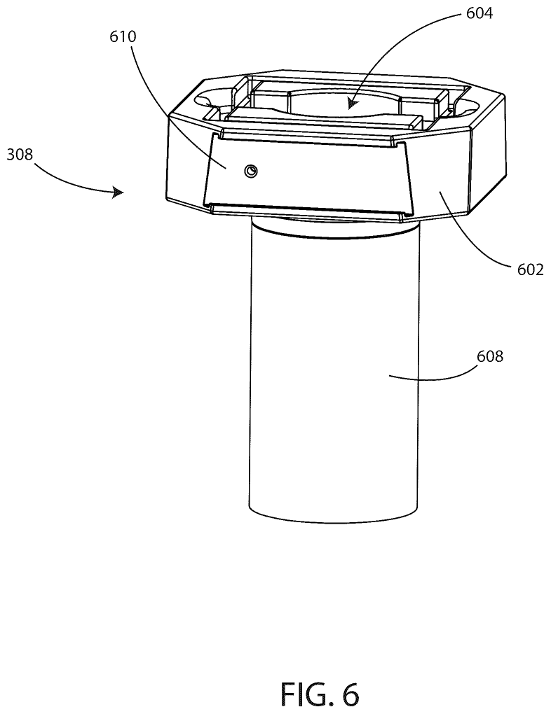

Referring now to FIG. 6, a perspective view of a rotatable component holder 308 is shown in accordance with various embodiments herein. The rotatable component holder 308 can include a top platform 602 or shelf structure and a cylindrical base 608. In various embodiments, the top platform 602 can be attached to a top of a cylindrical base 608 and can extend radially outward from the same. In some embodiments, the top platform 602 can extend radially outward at least about 0.5, 1, 2, 3, 4 centimeters (0.20, 0.39, 0.79, 1.18, 1.57 inches), or more from the cylindrical base 608. While the base 608 is cylindrical in this embodiment, it will be appreciated that it could also take on other forms in cross-section.

In various embodiments, the top platform 602 can be configured to rest upon or just over a top of an outer housing herein. In various embodiments, the cylindrical base 608 can be configured to pass through the top central aperture of the inner housing and the top central aperture of the outer housing.

The rotatable component holder 308 can also define one or more wells 604 on a top surface thereof. In various embodiments, the well 604 can be configured to receive and/or hold other components of the delivery system such as the mixing chamber, microsphere vial carrier and vial, the fluid injector and withdrawal assembly, etc.

In various embodiments, the catheter support arm 202 can be attached to the top platform 602 on a side (such as a flat side) thereof. In one approach, the rotatable component holder 308 can include a support arm receiving slot 610 or channel to facilitate attachment of the catheter support arm to the top platform 602 on a side thereof. However, it will be appreciated that the rotatable component holder 308 can also receive and/or retain the catheter support arm with structure other than a receiving slot 610 or channel.

FIG. 7 is a perspective view of a mounting bracket 340 in accordance with various embodiments herein. The mounting bracket 340 can facilitate mounting of the cancer therapy delivery assembly to a hospital bed, such as through attachment to the frame, rails, posts, head board, foot board, or other parts thereof. In some embodiments, the mounting bracket 340 can specifically facilitate mounting of the cancer therapy delivery assembly to a portion of a hospital bed meeting standard IEC 60601-2-52. The mounting bracket 340 can include a receiving plate 700 defining an area 702 to receive a base 302 of the system. The mounting bracket 340 can also include an attachment clip 704 to engage with a portion of a hospital bed or other structure that might be within a room where treatment is administered.

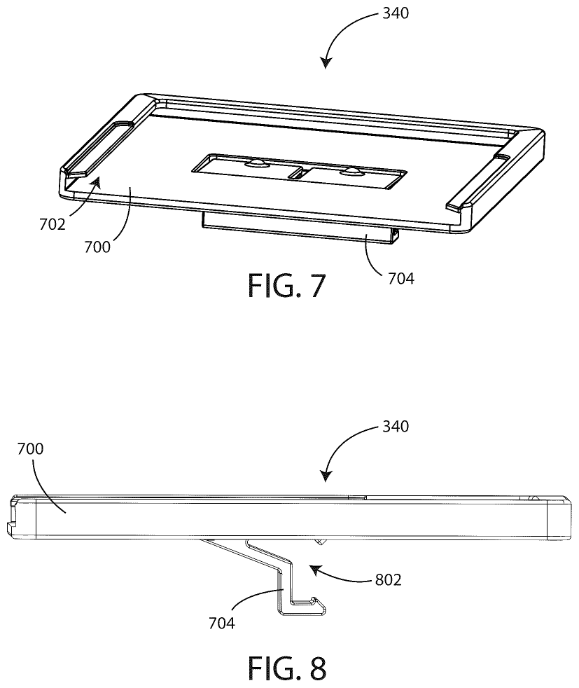

Referring now to FIG. 8, a side view is shown of a mounting bracket 340 in accordance with various embodiments herein. As before, the mounting bracket 340 can include a receiving plate 700 and an attachment clip 704. The attachment clip 704 can define a channel 802 to receive a portion of a hospital bed, such as rails thereof or another component. The mounting bracket 340 can serve to secure a system herein to a portion of a hospital bed. In some embodiments, more than one system can be secured to a hospital bed. For example, in some embodiments, multiple mounting brackets can be used to secure multiple systems herein to a hospital bed simultaneously, such as in the case of delivery of therapy to multiple different target sites within the body of a patient.

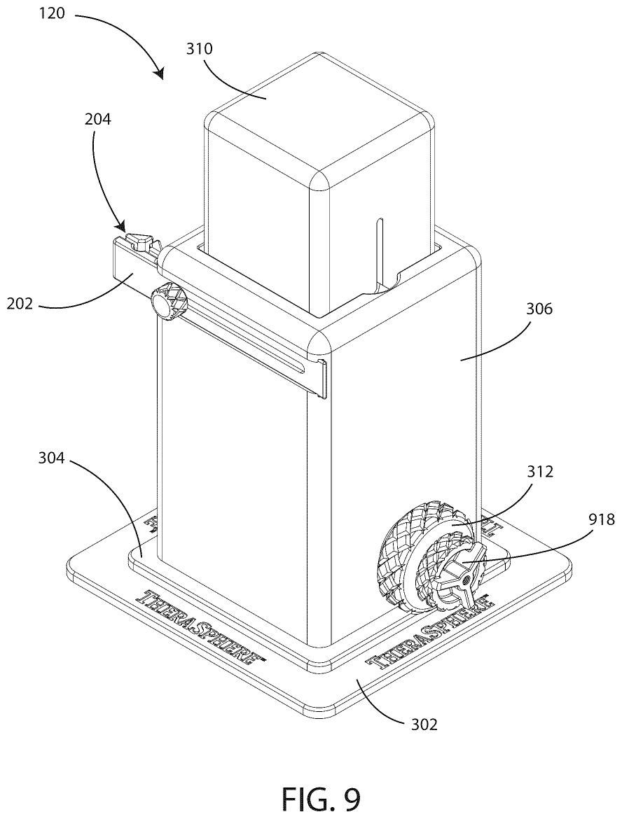

In some embodiments herein, a rotatable component holder can be omitted. For example, referring now to FIG. 9, a perspective view of a cancer therapy delivery assembly 120 is shown in accordance with various embodiments herein. As before, the cancer therapy delivery assembly 120 includes a catheter support arm 202, a catheter attachment element 204, a base 302, and inner housing 304, an outer housing 306, a transparent shielding member 310, and a vertical adjustment wheel 312. In this example, the cancer therapy delivery assembly 120 can also include a vertical position lock 918, to hold the vertical adjustment wheel 312 in a particular position. In this example, a fluid injector and withdrawal assembly and a radioactive microsphere supply reservoir-mixing chamber (not shown) can be disposed underneath the transparent shielding member 310, so as to contain radiation emitted from the therapeutic microspheres in the radioactive microsphere supply reservoir-mixing chamber.

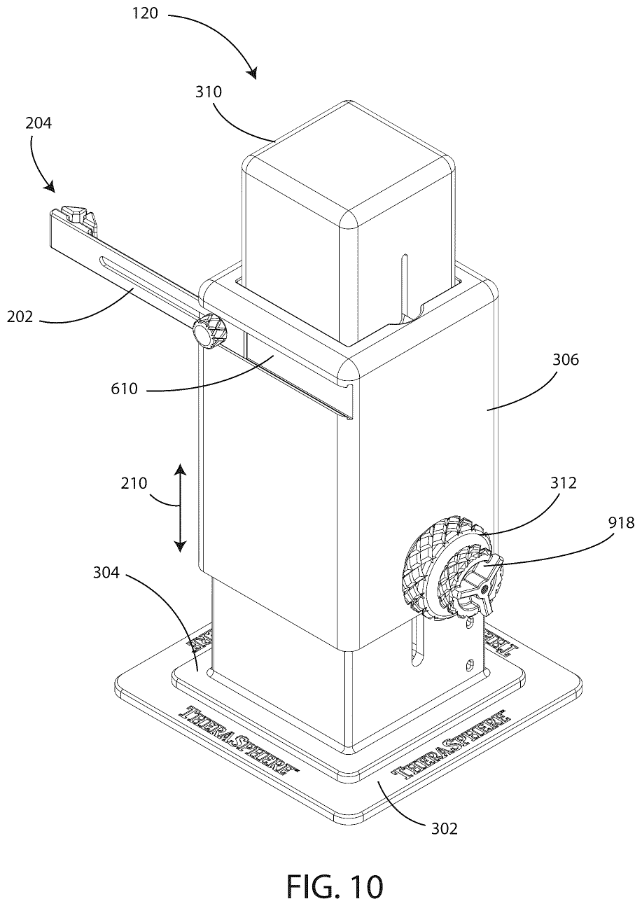

Referring now to FIG. 10, a perspective view of cancer therapy delivery assembly 120 is shown in accordance with various embodiments herein. FIG. 10 is generally similar to FIG. 9. As with FIG. 9, the cancer therapy delivery assembly 120 includes a catheter support arm 202, a catheter attachment element 204, a base 302, and inner housing 304, an outer housing 306, a transparent shielding member 310, and a vertical adjustment wheel 312. However, in the example of FIG. 10, the outer housing 306 has been moved vertically 210 with respect to the inner housing 304. In addition, the catheter support arm 202 (and the catheter attachment element 204 attached thereto) has been extended outward.

### Methods

Many different methods are contemplated herein, including, but not limited to, methods of making, methods of using, and the like. Aspects of system/device operation described elsewhere herein can be performed as operations of one or more methods in accordance with various embodiments herein.

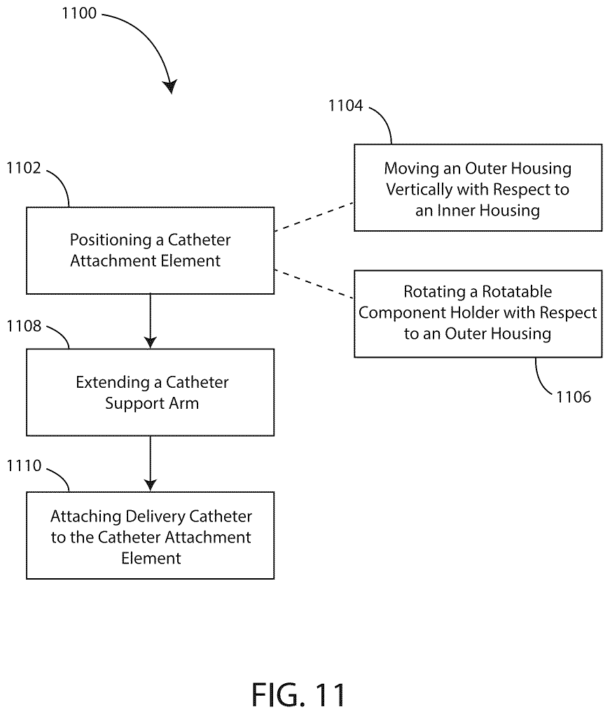

Referring now to FIG. 11, a flow diagram is shown illustrating operations of a method in accordance with various embodiments herein. The method can be a method of delivering a cancer therapy 1100. The method can include positioning a catheter attachment element as desired by a clinician 1102. For example, the method can include moving an outer housing vertically with respect to an inner housing 1104, to achieve vertical movement of the catheter attachment element. The method can also include rotating a rotatable component holder with respect to an outer housing to achieve rotational movement of the catheter attachment element 1106. The method can also include extending a catheter support arm with respect to a center of the cancer therapy delivery assembly to achieve radial movement of the catheter attachment element 1108. The method can also include attaching a cancer therapy delivery catheter to the catheter attachment element 1110. This operation can be performed either before or after positional adjustments are made to the catheter attachment element. The method can also include delivering therapeutic radioactive microspheres 1110 to a patient via the delivery catheter.

In an embodiment of the method, cancer therapy is delivered to treat a brain tumor. In an embodiment of the method, cancer therapy is delivered to treat glioblastoma. However, treatment of other types of tumors is also contemplated herein. By way of example, tumors and/or tissue of the head or neck, liver, breast, lung, pancreas, cervix, prostate, or eye, as well as other tissues can be treated in accordance with embodiments herein. In some embodiments it can be used to treat non-cancerous tumors or other tissue.

### Microspheres

Microspheres herein can include those with a combination of yttria, alumina, and silica. By way of example, in some embodiments, microspheres herein can include Y₂O₃—Al₂O₃—SiO₂ in a 40:20:40 wt. % ratio. It will be appreciated however, that other types of microspheres are also contemplated herein.

In some embodiments, microspheres can be prepared by combining yittrium-89 with alumina and silica, in some cases also using a flame spheroidization method, and using neutron bombardment to convert Y-89 into the beta emitting radioisotope Y-90. In various embodiments, the amount of beta radiation can exceed 2500, 3000, 4000, 5000, 6000, 7000, 8000, or even 9000 Bq per sphere at the time of activity calibration (recognizing that the amount of radiation will drop after that point as the Y-90 radioisotope decays). In some embodiments, the microspheres can be provided in a vial with activity of 3 GBq or lower up to 20 GB q or higher (at calibration time or “reference date and time”). However, in some embodiments, the microspheres can be provided in a vial with activity of less than 3, 2.75, 2.5, 2.25, 2, 1.75, 1.5, 1.25, 1.0, 0.75, 0.5, 0.4, 0.3, 0.35, 0.2, 0.15, or 0.1 GBq, or less at calibration time, or an amount falling within a range between any of the foregoing.

It will be appreciated that dosages can vary based on factors including the type of tumor/tissue to be treated, location of the tumor/tissue to be treated, factors specific to a particular patient, and the like. In some embodiments the dosage of the therapy can be less than or equal to 5000 Gy, 4500 Gy, 4000 Gy, 3500 Gy, 3000 Gy, 2500 Gy, 2000 Gy, 1500 Gy, 100 Gy, 500 Gy, 400 Gy, 300 Gy, 250 Gy, 225 Gy, 200 Gy, 180 Gy, 150 Gy, 120 Gy, 100 Gy, 90 Gy, 80 Gy, 70 Gy, 60 Gy, 50 Gy, 40 Gy, 30 Gy, or 20 Gy, or an amount falling within a range between any of the foregoing.

The size of the microspheres can be extremely small. In some embodiments, the average diameter of the microspheres can be from about 20 micrometers (μm) to about 30 μm. However, in some embodiments the microspheres can be somewhat smaller or larger.

The density of the microspheres can be quite high. In some embodiments, the density of the microspheres can be above 3 g/mL, such as from 3.1 to 3.5 g/mL, or about 3.3 g/mL. By comparison, the density of water at room temperature is about 0.9978 g/mL. As such, the density of microspheres is much higher than a saline solution which influences how readily such microspheres can settle out of a suspension.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

The invention claimed is:

1. A cancer therapy delivery assembly comprising:
a base;
an inner housing, wherein the inner housing is disposed over the base; and
an outer housing;
wherein the outer housing is disposed over the inner housing;
wherein the outer housing is moveable along a vertical axis relative to the inner housing to allow vertical height adjustment;
a rotatable component holder;
wherein the rotatable component holder is disposed on the outer housing;
wherein the rotatable component holder is configured to rotate relative to the outer housing; and
a transparent shielding member, wherein the transparent shielding member is disposed over the rotatable component holder.

2. The cancer therapy delivery assembly of claim 1, further comprising a vertical position adjustment device, wherein manipulation of the vertical position adjustment device causes the outer housing to move along a vertical axis relative to the inner housing.

3. The cancer therapy delivery assembly of claim 2, the vertical position adjustment device comprising an adjustment wheel.

4. The cancer therapy delivery assembly of claim 1, further comprising a rotational lock element, wherein the rotational lock element is configured to lock the rotatable component holder in a particular rotational position.

5. The cancer therapy delivery assembly of claim 1,
the inner housing comprising a top central aperture;
the outer housing comprising a top central aperture; and
the rotatable component holder comprising a cylindrical base, wherein the cylindrical base is configured to pass through the top central aperture of the inner housing and the top central aperture of the outer housing.

6. The cancer therapy delivery assembly of claim 5, the rotatable component holder further comprising a top platform, wherein the top platform is attached to a top of the cylindrical base and extends radially outward from the same.

7. The cancer therapy delivery assembly of claim 6,
wherein the top platform is configured to rest upon a top of the outer housing;
the cancer therapy delivery assembly further comprising a catheter support arm;
wherein the catheter support arm is attached to the rotatable component holder; and
wherein the catheter support arm is configured to adjustably move inward or outward with respect to a center of the cancer therapy delivery assembly.

8. The cancer therapy delivery assembly of claim 7, the catheter support arm comprising a catheter attachment element, wherein the catheter attachment element is disposed on an end of the catheter support arm.

9. The cancer therapy delivery assembly of claim 1, the rotatable component holder defining a well, wherein the well is configured to receive a vial holder and a vial with radioactive microspheres.

10. The cancer therapy delivery assembly of claim 1, the outer housing comprising one or more exterior facets.

11. The cancer therapy delivery assembly of claim 1, the outer housing comprising a substantially polygonal perimeter in cross-section.

12. The cancer therapy delivery assembly of claim 1, wherein the outer housing is disposed over the inner housing in a nested configuration.

13. The cancer therapy delivery assembly of claim 1, the transparent shielding member defining one or more slots to allow passage of a fluid conduit therethrough.

14. A cancer therapy delivery assembly comprising:
a base;
an inner housing, wherein the inner housing is disposed over the base; and
an outer housing;
wherein the outer housing is disposed over the inner housing;
wherein the outer housing is moveable along a vertical axis relative to the inner housing;
a transparent shielding member, wherein the transparent shielding member is disposed over the outer housing; and
a vertical position adjustment device, wherein manipulation of the vertical position adjustment device causes the outer housing to move along a vertical axis relative to the inner housing.

15. The cancer therapy delivery assembly of claim 14, the outer housing defining a well, wherein the well is configured to receive a vial holder and a vial with radioactive microspheres.

16. The cancer therapy delivery assembly of claim 14, the outer housing comprising one or more exterior facets.

17. The cancer therapy delivery assembly of claim 14, the outer housing comprising a substantially polygonal perimeter in cross-section.

18. The cancer therapy delivery assembly of claim 14, wherein the outer housing is disposed over the inner housing in a nested configuration.

19. The cancer therapy delivery assembly of claim 14, further comprising a catheter support arm;
wherein the catheter support arm is attached to the outer housing; and
wherein the catheter support arm is configured to adjustably move inward or outward with respect to a center of the cancer therapy delivery assembly.

20. A method of delivering a cancer therapy comprising:
positioning a catheter attachment element, the positioning comprising
moving an outer housing vertically with respect to an inner housing;
extending a catheter support arm radially; and
rotating a rotatable component holder with respect to the outer housing;
attaching a cancer therapy delivery catheter to the catheter attachment element; and
delivering therapeutic radioactive microspheres to a patient via the delivery catheter.
